(19) United States
(12) Patent Application Publication
(10) Pub. No.: US 2026/0137295 A1
(43) Pub. Date: May 21, 2026

(54) IMAGING SLOW FLOW DYNAMICS OF CEREBROSPINAL FLUID USING MAGNETIC RESONANCE IMAGING

(71) Applicant: The General Hospital Corporation, Boston, MA (US)

(72) Inventors: Zijing Dong, Charlestown, MA (US); Fuyixue Wang, Charlestown, MA (US); Lawrence L. Wald, Cambridge, MA (US); Laura Lewis, Cambridge, MA (US); Jonathan Polimeni, Cambridge, MA (US)

(21) Appl. No.: 19/119,706

(22) PCT Filed: Oct. 12, 2023
(86) PCT No.: PCT/US2023/076679

§ 371 Date: Apr. 9, 2025

Related U.S. Application Data
(60) Provisional application No. 63/415,583, filed on Oct. 12, 2022.

(51) Int. Cl. A61B 5/055; A61B 5/00

(52) U.S. Cl. CPC A61B 5/055; A61B 5/4064; A61B 5/4076; A61B 5/7289

(57) ABSTRACT

Magnetic resonance imaging (MRI) is used to image the slow flow of cerebrospinal fluid (CSF) in subarachnoid and perivascular spaces, amongst other regions of the brain and central nervous system. The slow CSF flow imaging can be provided by using low velocity’ encoding (VENC) and an efficient data acquisition. Tailored image processing can be used to further improve the accuracy, specificity, and visualization of CSF flow dynamics.

### CROSS-REFERENCE TO RELATED APPLICATIONS

This application claims the benefit of U.S. Provisional Patent Application Ser. No. 63/415,583, filed on Oct. 12, 2022, and entitled “IMAGING SLOW FLOW DYNAMICS OF CEREBROSPINAL FLUID IN SUBARACHNOID AND PERIVASCULAR SPACES USING MRI,” which is herein incorporated by reference in its entirety.

### STATEMENT OF FEDERALLY SPONSORED RESEARCH

This invention was made with government support under EB030006, EB019437, AT011429, and NS123717 awarded by the National Institutes of Health. The government has certain rights in the invention.

### BACKGROUND

Cerebrospinal fluid (CSF) flow is a key component of the brain's waste clearance system which is impaired in many neurological disorders, such as Alzheimer's disease. However, both CSF flow dynamics and how they link to brain function/physiology are not well understood, especially in the subarachnoid space (SAS) surrounding the cerebrum and in perivascular space (PVS) surrounding blood vessels within the tissue. MRI is a useful non-invasive tool for CSF flow measurement, and different contrast mechanisms have been investigated to provide complementary information. Among them, velocity-encoded phase-contrast (PC) methods can directly measure coherent bulk flow of the CSF, and can provide comprehensive information of the flow dynamics, including its velocity, direction, and net flow. However, most studies using PC methods applied to measuring CSF flow focused on brain regions with relatively fast CSF flow such as ventricles and aqueduct, due to the challenges to map the slow flow in the SAS and PVS regions, such as low sensitivity, physiological noise in the phase-valued image data, and signal dephasing. The lack of imaging method to study slow CSF flow in SAS and PVS limits our understanding of the brain's clearance pathway and the underlying mechanism of many neurological diseases.

### SUMMARY OF THE DISCLOSURE

The present disclosure addresses the aforementioned drawbacks by providing a method for cerebrospinal fluid (CSF) flow imaging with a magnetic resonance imaging (MRI) system. The method includes acquiring magnetic resonance data from a subject with an MRI system, where the magnetic resonance data are acquired using a pulse sequence having velocity encoding gradients sufficient to encode slow flow of the CSF in the subject. Phase contrast images are reconstructed from the magnetic resonance data using a computer system, and velocity images are generated from the phase contrast images. The velocity images are indicative of the slow flow of the CSF in the subject. CSF flow data are then generated from the velocity images using the computer system, where the CSF flow data are indicative of at least one of flow velocity magnitude, flow velocity direction, or flow volume of the CSF in the subject. The CSF flow data are output using the computer system.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 is a pulse sequence diagram for an example pulsed-gradient spin-echo (PGSE) pulse sequence that can be used to acquire velocity-encoded magnetic resonance data.

FIG. 2 is a flowchart setting forth the steps of an example method for slow cerebrospinal fluid (CSF) flow imaging in accordance with some embodiments described in the present disclosure.

FIG. 3 shows an example results acquired using the systems and methods described in the present disclosure. The time series of the flow velocity along the z-direction in the selected region of interest within the subarachnoid space shows clear periodic respiration-driven fluctuations, and the corresponding frequency spectrum peaks at 0.1 Hz, which is the frequency of the paced breathing task.

FIG. 4 shows example velocity maps (along z-direction) of the CSF at two selected cardiac phases (systole and diastole). Three ROIs were selected, and their corresponding velocities are plotted as a function of cardiac phase.

FIG. 5 shows example CSF flow vector field maps generated using the disclosed systems and methods with whole-brain coverage. The direction of 3D flow vector projected onto this plane is indicated by the vector field with each vector visualized with a normalized length. The flow direction changes (highlighted by white arrows) between diastole and systole can be observed in subarachnoid space.

FIG. 6 shows three selected ROIs (left), the corresponding time series of the flow velocity along the z-direction in the ROIs (middle), and the corresponding frequency spectrum of the flow velocity in each ROI (right).

FIG. 7 shows another example of velocity maps (along z-direction) at two selected cardiac and respiratory phases (top panel). Three ROIs were selected, and their corresponding velocity were plotted as a function of cardiac and respiratory phase.

FIG. 8 shows another example of velocity vector maps in midsagittal view at two selected cardiac phases. The direction of flow projected onto this plane is shown by color-coded vectors. The flow direction changes (highlighted by white arrows) between diastole and systole are consistent across subjects.

FIG. 9 is a block diagram of an example MRI system that can implement the methods described in the present disclosure.

### DETAILED DESCRIPTION

Described here are systems and method for imaging cerebrospinal fluid (CSF) using magnetic resonance imaging (MRI). Advantageously, the disclosed systems and methods are capable of imaging the slow flow of CSF in subarachnoid and perivascular spaces. In general, the slow CSF flow imaging can be provided by using low velocity encoding (VENC) and an efficient data acquisition. Additionally or alternatively, tailored image processing can be used to further improve the accuracy, specificity, and visualization of CSF flow dynamics.

As one advantage, the disclosed systems and methods for slow CSF flow imaging can provide a useful tool to study the flow dynamics and patterns in the subarachnoid and perivascular spaces. By analyzing these flow dynamics and patterns their association with brain function and physiology can be assessed. As another advantage, imaging the slow CSF flow can provide for monitoring and otherwise assessing dysfunction of the waste clearance system of the human brain. As a result, the disclosed systems and methods can be used to find new biomarkers for many neurological disorders, such as Alzheimer's disease.

In general, the disclosed systems and methods implement a CSF flowmetry technique that is based on phase-contrast MRI. The disclosed imaging framework allows for measurements of the slow flow in neuroanatomical regions (e.g., the subarachnoid space, perivascular spaces) with high sensitivity, spatiotemporal resolution, and wide spatial coverage. In this way, the disclosed systems and methods allow for the investigation of flow dynamics across the whole brain and can provide further insight into regions with slow flow.

Using the slow CSF flow imaging techniques described herein, CSF flow data can be obtained from magnetic resonance image data. The flow information in the obtained CSF flow data can be four-dimensional (4D), including three spatial dimensions and one temporal dimension. The magnitude of the flow velocity, direction, and net flow can be obtained from the acquired image data and stored as part of the CSF flow data.

The general slow CSF flow imaging framework can include some or all of the following components. In one aspect, a pulse sequence that can achieve low VENC with minimized signal loss and high acquisition efficiency (e.g., an echo planar imaging (EPI) readout) can be used to acquire magnetic resonance data, from which images are reconstructed. Postprocessing can be used to remove background phase variations (e.g., related to noise and artifacts) from the acquired data. Application-dependent processing can then be used to generate CSF flow maps (e.g., magnitude flow maps, direction flow maps, flow volume maps) and/or its dynamic change with specific brain physiological or functional activities.

The disclosed systems and methods enable imaging of the slow CSF flow in the human brain non-invasively, with high sensitivity, efficiency, spatiotemporal resolution, and coverage. As noted above, the developed slow CSF flow imaging method can provide a useful tool to study the flow dynamics and patterns in the subarachnoid and perivascular spaces, and their association with brain function and physiology (e.g., cardiac pulsation, respiration), which have been previously inaccessible. For example, the response and flow dynamics under different functional activations (e.g., generated by presenting visual or auditory stimuli), interventions (exercise), and arousal states (awake, sleep) can be investigated using the disclosed systems and methods.

Advantageously, the disclosed systems and methods can also be applied to measuring slow flow in other parts of the brain, including the slow blood flow in the small blood vessels such as parenchymal or penetrating arteries and veins. Moreover, imaging the slow CSF flow in the human brain can enable monitoring and/or assessing dysfunction of the brain's waste clearance system: to facilitate the search of new biomarkers (e.g., global or regional flow change between health and disease population) for many neurological disorders, such as Alzheimer's disease: and to evaluate the effectiveness or efficacy of treatments.

As one non-limiting example, a pulsed-gradient-spin-echo (PGSE) pulse sequence can be used to achieve an efficient low-VENC acquisition. The PGSE pulse sequence provides several advantages for slow CSF flow imaging. As one example, the PGSE pulse sequence enables fast sampling provided by EPI readouts used when acquiring k-space data. As another example, snapshot velocity encoding can be implemented by using a single-shot image acquisition. By using a single-shot acquisition, shot-to-shot variations can be avoided. As still another example, the PGSE pulse sequence is more robust to physiological noise in the phase-valued image data (e.g., breathing-related B₀ field variation) when using a spin-echo over gradient-echo acquisition. The PGSE pulse sequence also has the flexibility to achieve low VENC (e.g., 1 cm/s or lower), which can be used to measure slow flow, because the PGSE pulse sequence allows for long velocity encoding time (or pulse time interval, Δ) and therefore smaller gradient amplitude, thereby reducing undesirable strong diffusion weighting and associated signal loss. Additionally, the strong T2-weighting from long echo time (TE) values in the PGSE pulse sequence reduces the signal from blood or parenchyma, which can reduce partial volume effects and improve specificity to CSF.

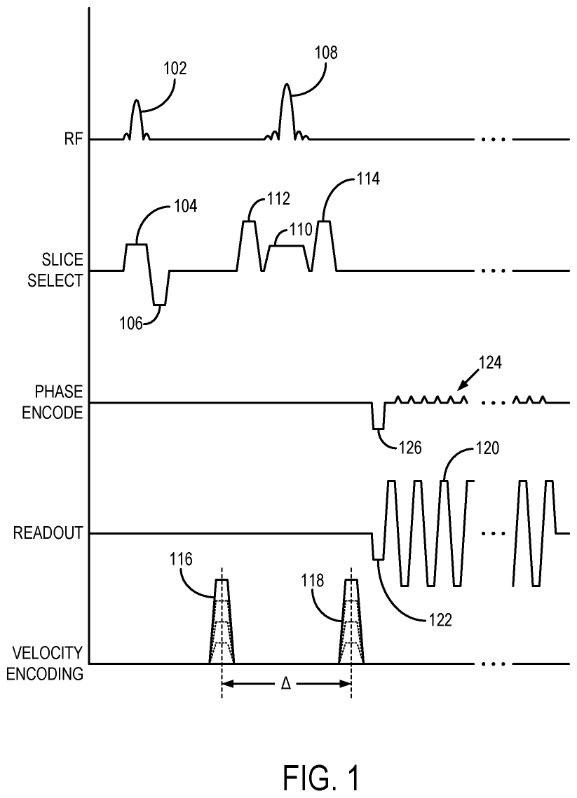

An example of a PGSE pulse sequence that can be used in some embodiments described in the present disclosure is shown in FIG. 1. The pulse sequence includes a radio frequency (RF) excitation pulse 102 that is played out in the presence of a slice-select gradient 104 in order to produce transverse magnetization in a prescribed imaging slice. The slice-select gradient 104 includes a rephasing lobe 106 that acts to rephase unwanted phase dispersions introduced by the slice-select gradient 104, such that signal losses resultant from these phase dispersions are mitigated. Next, a refocusing RF pulse 108 is applied in the presence of another slice select gradient 110 in order to refocus transverse spin magnetization. In order to reduce unwanted phase dispersions, a first crusher gradient 112 bridges the slice select gradient 110 with a second crusher gradient 114. The crusher gradients 112, 114 are optional and can be removed to reduce their effect on velocity encoding.

The slice select gradient 110 and crusher gradients 112 and 114 are further bridged by first and second velocity-encoding gradients, 116 and 118, respectively. These velocity-encoding gradients 116 and 118 are equal in size, that is, their areas are equal. The velocity-encoding gradients 116 and 118, while shown on a separate “velocity encoding” gradient axis, are in fact produced through the application of velocity-encoding gradient lobes along one or more of the slice-encoding, phase-encoding, and frequency-encoding (e.g., readout) gradient directions. By changing the amplitudes and other characteristics of the velocity-encoding gradient lobes, the acquired echo signals can be weighted for flow velocities along any arbitrary direction. For example, when the velocity-encoding weighting gradients 116 and 118 are composed solely of gradient lobes applied along the G_{z} gradient axis, the acquired echo signals will be weighted for flow velocities occurring along the z-direction. As another example, if the diffusion weighting gradients 116 and 118 are composed of gradient lobes applied along both the Gₓ and G_{y} gradient axes, then the echo signals will be weighted for flow velocities occurring in the x-y plane along a direction defined by the relative amplitudes of the gradient lobes.

Velocity encoding of the acquired echo signals is provided when spins move along the direction of the velocity-encoding direction during the time interval, Δ, spanned between the application of the first and second velocity-encoding gradients 116 and 118, respectively. The first velocity-encoding gradient 116 dephases the spins in the imaging volume, whereas the second velocity-encoding gradient 118 acts to rephase the spins by an equal amount. Spins that are stationary during the time interval, Δ, so not receive a net phase accumulation, while spins that are moving along the velocity-encoding direction will receive a net phase accumulation that is proportional to the velocity of the spins.

Following excitation of the nuclear spins in the prescribed imaging volume, data are acquired by sampling a series of velocity-encoded echo signals in the presence of an alternating readout gradient 120. The alternating readout gradient 120 is preceded by the application of a pre-winding gradient 122 that acts to move the first sampling point along the frequency-encoding, or readout, direction by a prescribed distance in k-space. Spatial encoding of the echo signals along a phase-encoding direction is performed by a series of phase encoding gradient “blips” 124, which are each played out in between the successive signal readouts such that each echo signal is separately phase-encoded. The phase-encoding gradient blips 124 are preceded by the application of a pre-winding gradient 126 that acts to move the first sampling point along the phase-encoding direction by a prescribed distance in k-space.

As is known in the art, the foregoing pulse sequence can be repeated a plurality of times while applying a different slice select gradients 104 and 110 during each repetition such that a plurality of slice locations are imaged.

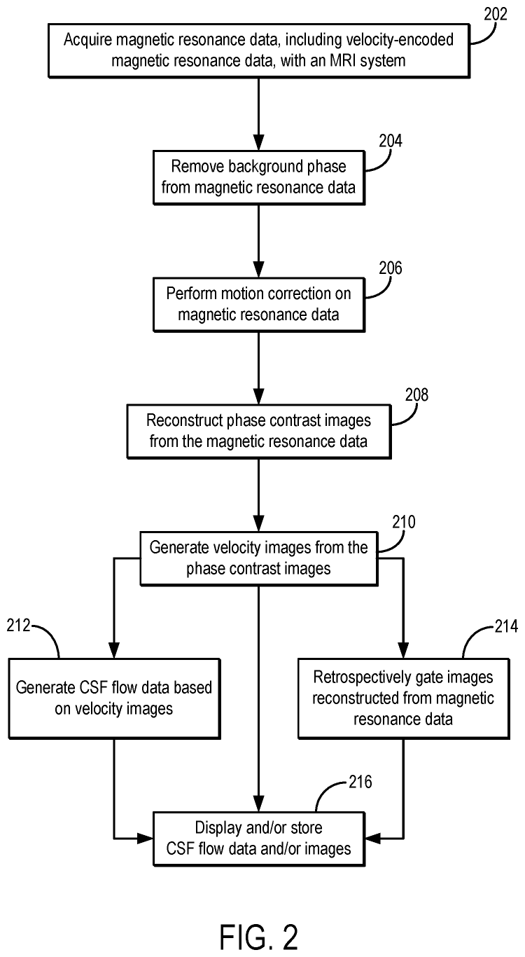

Referring now to FIG. 2, a flowchart is illustrated as setting forth the steps of an example method for slow CSF flow imaging with an MRI system. The method includes acquiring magnetic resonance data with an MRI system, as indicated at step 202. As one example, the magnetic resonance data are velocity-encoded (or flow-encoded) magnetic resonance data acquired using a slow-flow phase-contrast data acquisition having a low-VENC encoding and efficient EPI readout. As described above, in one non-limiting example a PGSE pulse sequence, such as the one illustrated in FIG. 1, can be used. In addition, multi-VENC acquisitions can be employed to improve the sensitivity and velocity range of the acquired data to allow mapping of slow flow along with potentially faster flows (e.g., flow within ventricles). For instance, multi-VENC acquisitions can be implemented by changing the magnitude of the velocity-encoding gradients in subsequent acquisitions such that the acquired magnetic resonance data are sensitized to flows with different velocity magnitudes (e.g., slow flow versus faster flows).

In addition to PGSE, other acquisition methods can also be used to achieve a low-VENC acquisition to map slow CSF flow. In these instances, additional or different postprocessing specific to the acquisition used can be implemented. For example, a gradient-echo EPI sequence is also an efficient acquisition and increasing the gradient amplitude and the pulse time interval can provide lower VENC in a gradient-echo EPI pulse sequence. The gradient-echo acquisition can be more sensitive to physiological-related phase variations, signal loss due to stronger velocity gradient if a short TE value is desired, or T2* dephasing and signal voids when using long TE; therefore, higher-order phase removal and protocol optimization to minimize these effects can be used when implementing a gradient-echo EPI pulse sequence rather than PGSE. Multi-echo pulse sequences can also be used to improve the background phase removal and to help with increased sensitivity and specificity. A multi-echo pulse sequence can be implemented using either gradient-echo or spin-echo-based acquisitions. Additionally or alternatively, non-Cartesian sampling patterns (e.g., spiral, radial, PROPELLER) can be used instead of a Cartesian sampling pattern (e.g., EPI) for an efficient imaging readout.

In some implementations, additional magnetic resonance data can be acquired using different imaging techniques. For example, in addition to slow flow imaging using phase-contrast MRI, data can be acquired with other imaging contrasts. For example, diffusion-contrast, derived from the magnitude-valued data, can be used to map the level of incoherence of the flow (diffusion contrast, intravoxel dephasing) concurrently with or alongside the phase-valued data used to map the coherent bulk flow (using phase contrast as described above), which can provide complementary and thus more comprehensive information of the slow CSF flow dynamics.

Additionally, in some implementations the magnetic resonance data are acquired while the subject is performing a functional task, or otherwise under engaging in a functional activation. For example, the magnetic resonance data may be acquired while the subject is being present a visual and/or auditory stimulus. Alternatively, the magnetic resonance data can be acquired while the subject is performing an intervention (e.g., exercise) or in a particular arousal state (e.g., awake, asleep). In these instances, the response and flow dynamics under these different functional activations may be analyzed, as noted below.

Background phase offset is removed from the magnetic resonance data as indicated at step 204. For instance, to minimize the effect of phase variations caused by systemic physiology and other nuisance factors, background phase removal can be performed by removing the spatially low-polynomial-order phase of each dynamic independently. As one non-limiting example, an optimized coil combination for the phase value image can be used to ensure the quality of the phase images and avoid artifacts.

In some implementations, motion correction may be performed on the magnetic resonance data, as indicated at step 206. For instance, bulk motion of the subject (e.g., subject movement during scanning, respiration, cardiac motion) can be measured or otherwise estimated and the effects of these motions can be corrected for in the magnetic resonance data. As an example, motion measurements can be obtained from external devices, such as respiratory belts, pulse oximeters, ECG leads, and so on.

Phase contrast images are then reconstructed from the magnetic resonance data, as indicated at step 208. Velocity images are then generated from the reconstructed phase contrast images, as indicated at step 210. For instance, the phase information in the phase contrast images may be converted to velocity image data.

Flow data are then generated based on the velocity images, as indicated at step 212. As noted above, the CSF flow data may include slow CSF flow data that indicate slow flow dynamic of CSF in the brain or other neuroanatomical locations. The CSF flow data may include flow maps, which may include velocity magnitude maps, velocity direction maps, flow volume maps, net flow velocity maps, net flow volume maps, and the like.

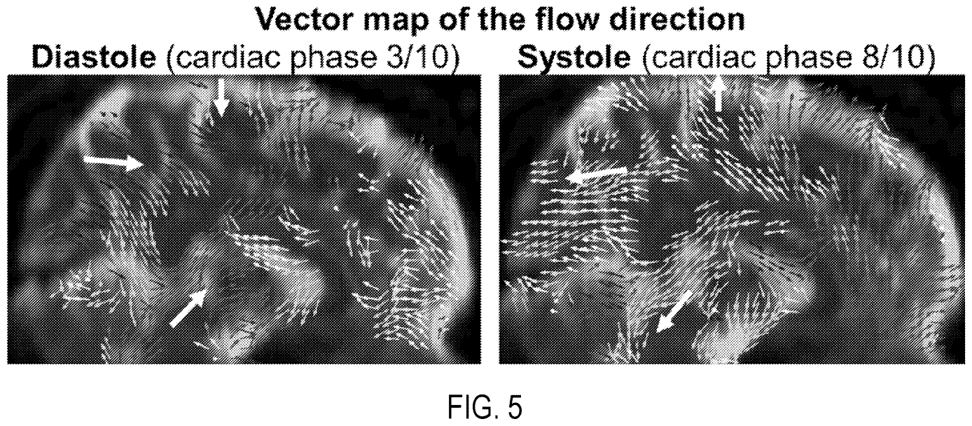

For example, velocity direction mapping can be performed to generate velocity direction maps. In this instance, the velocity images may be processed using a velocity direction analysis with multi-direction encoding to perform the velocity direction mapping. The direction of the CSF flow of each voxel can also be obtained by acquiring multiple encoding directions (e.g., x, y, z). In addition, to improve the visualization of the flow information, especially the flow direction information, vector fields (e.g., FIG. 5) or streamline maps can be calculated and generated from multi-directional CSF flow datasets.

The CSF flow data provide quantitative estimation of the CSF flow, which can be either absolute or relative velocities depending on the acquisition schemes and estimation method. The estimation of absolution velocity utilizes reference data without velocity encoding, which can be acquired in step 202.

When data are acquired under different functional activations, as described above, the CSF flow data will be indicative of the response and/or flow dynamics associated with the functional activation performed (e.g., generated by presenting visual or auditory stimuli), interventions (exercise), or arousal states (awake, sleep). In these instances, the CSF flow data can indicate, for example, changes in flow dynamics that are associated with the functional activation, intervention, or arousal state. By monitoring these changes in CSF flow dynamics under different conditions, further insight into functional neuroanatomy can be realized.

As noted above, the CSF flow data can be indicative of slow CSF flow in the human brain. As a result, the analysis of the CSF flow data can enable monitoring and/or assessing dysfunction of the brain's waste clearance system; can facilitate searching for biomarkers (e.g., global or regional flow change between health and disease population) for many neurological disorders, such as Alzheimer's disease; and can be used to evaluate the effectiveness or efficacy of treatments.

Additionally or alternatively, images reconstructed from the magnetic resonance data may be retrospectively gated based on the velocity images, as indicated at step 214. For instance, flow dynamic across specific physiological and/or functional processes may be tracked in the velocity images, which may then be used to provide retrospective gating. For 4D CSF flow imaging, to study the flow dynamics across specific physiological and/or functional processes, retrospective gating can be employed by grouping each slice or shot based on the cardiac or respiratory phase at the time of the slice acquisition. With gating, multi-shot pulse sequences (e.g., multi-shot EPI, multi-shot GRE) can be used to acquire the velocity-encoded data with good accuracy by avoiding large shot-to-shot variations due to the differences in physiological states. In addition to retrospective gating, prospective gating approaches can also be used to acquire images and generate velocity images for the corresponding physiological status (e.g., cardiac or respiratory cycles).

The generated images and/or CSF flow data may then be displayed to a user, or stored for later use or processing, as indicated at step 216.

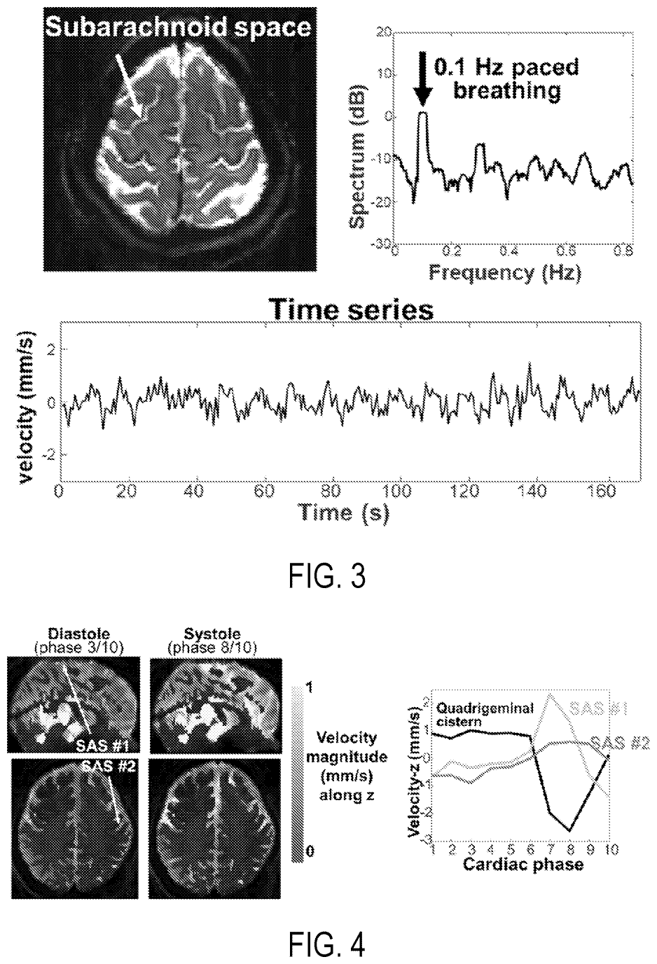

In an example study, the disclosed systems and methods were used to measure slow CSF flow in the subarachnoid space of the human brain. FIG. 3 shows the results of high temporal resolution scans, where clear respiration-associated periodic velocity changes are observed in the subarachnoid space with an amplitude of ˜1 mm/s. The frequency spectra also show high energy at 0.1 Hz, corresponding to the frequency of a paced breathing task that was performed during imaging in this experiment. FIG. 4 shows example maps and time-series plots of flow velocity along the z-direction (head-to-foot) across cardiac cycles, which were generated with retrospective gating. As can be seen, in the subarachnoid space, strong and rapid CSF flow changes occur across cardiac phases (e.g., upward/downward flow during diastole/systole). Moreover, different time-delays of the velocity change are also observed in different subarachnoid space regions. Using 3-directional velocity encoding, directions of CSF flow across the whole brain were obtained, and a projection of the 3D vector field (normalized vector length) onto a 2D sagittal slice is presented in FIG. 5. The flow directions in the ventricles are consistent with previous literature (e.g., inflow into the brain during diastole and outflow during systole). Apparent and coherent direction changes of the subarachnoid space flow were observed.

In another example, data were acquired on a 7 T MRI scanner with a 64-channel head array coil on healthy subjects (N=4). A PGSE sequence was used to acquire the phase contrast images for: i) fast sampling provided by EPI readouts; ii) snapshot velocity encoding with a single-shot acquisition, thereby avoiding shot-to-shot variations; iii) higher robustness to physiological noise in the phase (e.g., breathing-related B₀ field variation) when using spin-echo over gradient-echo; iv) flexibility to achieve low VENC (1 cm/s or lower) for measuring slow flow, since it allows for long velocity encoding time (or pulse time interval, Δ) and therefore smaller gradient amplitude, reducing undesirable high diffusion contrast and associated signal loss; and v) strong T2 weighting from long TE values that reduces the signal from blood or parenchyma for improved specificity to CSF. An ASPIRE coil combination was used to improve the quality of the phase-valued images. To minimize the effect of phase variations caused by systemic physiology and other nuisance factors, background phase removal was performed by removing the spatially low-polynomial-order phase of each dynamic independently, without any temporal filtering. For whole-brain 4D CSF flow imaging, retrospective gating was employed by grouping each EPI slice based on the cardiac or respiratory phase at the time of the slice acquisition.

High temporal resolution scans in the fourth ventricle and subarachnoid space were acquired with a paced breathing task (0.1 Hz, 5 s breathe in, and 5 s breathe out). To investigate real-time velocity and its frequency spectrum, short TR values (450 ms for fourth ventricle scan, 600 ms for subarachnoid space scan) were used with small coverage (4 slices). Other acquisition parameters were: resolution=1.5×1.5×5 mm3, VENC=3 cm/s for the fourth ventricle scan, and 1 cm/s for the subarachnoid space scan. Whole-brain 4D CSF flow imaging data were acquired in 3 healthy subjects with paced breathing tasks. Cardiac and respiratory cycles were recorded using external sensors for retrospective gating. Three velocity encoding directions (x, y, z) were acquired using the following parameters: resolution=1.5×1.5×3 mm3, 36 slices, VENC=1 cm/s, TR/TE=2100/83 ms, MB=2, scan of each run lasted ˜4 minutes, thus 3 directions required ˜12 minutes.

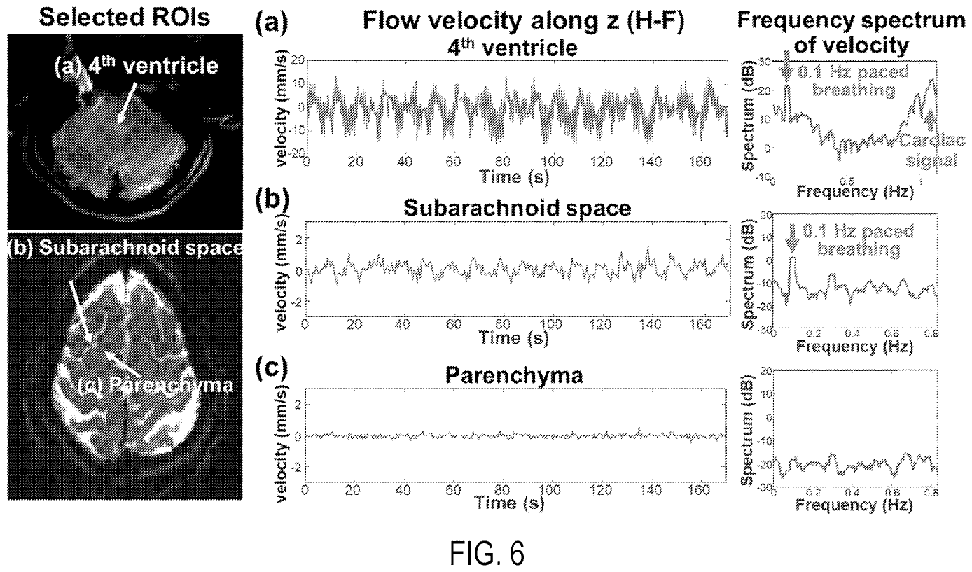

FIG. 6 shows the results of the high temporal resolution scans, where clear respiration-associated periodic velocity changes are observed in both the 4th ventricle (FIG. 6 (a)) with an amplitude of ˜10 mm/s and in SAS (FIG. 6 (b)) with ˜1 mm/s. The frequency spectra also show high energy at 0.1 Hz, corresponding to the frequency of the paced breathing. This is not detected in a control ROI within the parenchyma (FIG. 6 (c)), indicating that the observed periodic change is not noise due to breathing-related background phase variations. In addition to respiration, strong modulation from cardiac pulsation also appears in the time series as rapid peaks riding on the slower, periodic respiratory waveform.

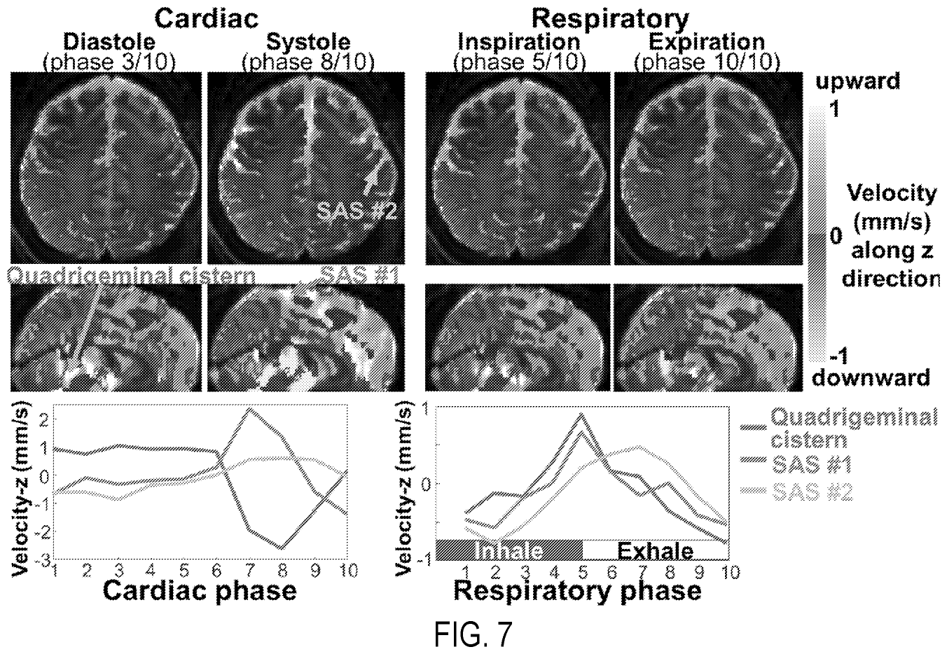

FIG. 7 shows example maps and time-series plots of flow velocity along z-direction (head-to-foot) across cardiac and respiratory cycles. As can be seen, in both ventricles and subarachnoid space, strong and rapid CSF flow changes occur across cardiac phases (e.g., upward/downward flow during diastole/systole in the cistern and ventricles), while relatively weaker (smaller amplitude) and slower changes are observed with the respiratory cycle. Moreover, different time-delays of the velocity change are also observed in different SAS regions (FIG. 7 (bottom)).

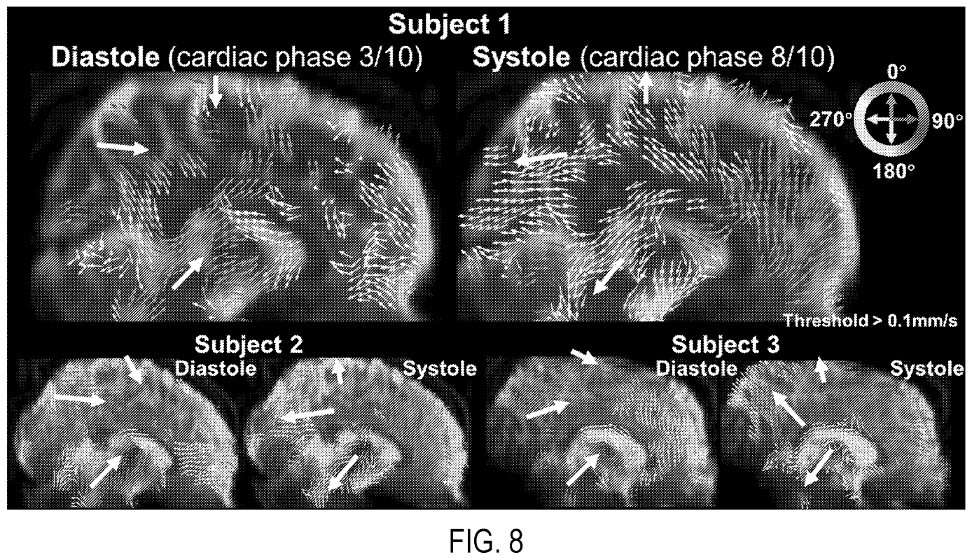

Using 3-directional velocity encoding, directions of CSF flow across the whole brain were obtained, and a projection of the 3D vector field (normalized vector length, color-coded direction) onto a 2D sagittal slice is presented in FIG. 8. The flow directions in the ventricles are consistent with previous literature (e.g., inflow into the brain during diastole and outflow during systole). Apparent and coherent direction changes of the subarachnoid space flow were approximately consistent across subjects.

The described use of 4D CSF flowmetry using phase contrast (PC)-PGSE provides high sensitivity and efficiency to study the flow dynamics in ventricles, subarachnoid space, and/or perivascular space. Preliminary data indicate that both cardiac pulsation and respiration drive CSF flow in ventricles and subarachnoid space, and cardiac pulsation introduces stronger and more rapid CSF flow changes than respiration in most areas.

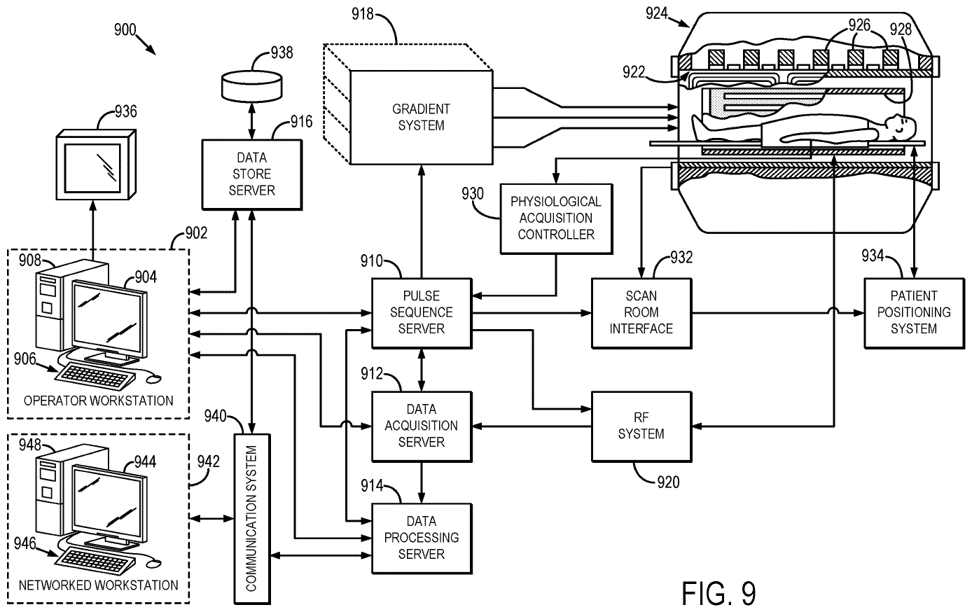

Referring particularly now to FIG. 9, an example of an MRI system 900 that can implement the methods described here is illustrated. The MRI system 900 includes an operator workstation 902 that may include a display 904, one or more input devices 906 (e.g., a keyboard, a mouse), and a processor 908. The processor 908 may include a commercially available programmable machine running a commercially available operating system. The operator workstation 902 provides an operator interface that facilitates entering scan parameters into the MRI system 900. The operator workstation 902 may be coupled to different servers, including, for example, a pulse sequence server 910, a data acquisition server 912, a data processing server 914, and a data store server 916. The operator workstation 902 and the servers 910, 912, 914, and 916 may be connected via a communication system 940, which may include wired or wireless network connections.

The pulse sequence server 910 functions in response to instructions provided by the operator workstation 902 to operate a gradient system 918 and a radiofrequency (“RF”) system 920. Gradient waveforms for performing a prescribed scan are produced and applied to the gradient system 918, which then excites gradient coils in an assembly 922 to produce the magnetic field gradients Gₓ, G_{y}, and G_{z} that are used for spatially encoding magnetic resonance signals. The gradient coil assembly 922 forms part of a magnet assembly 924 that includes a polarizing magnet 926 and a whole-body RF coil 928.

RF waveforms are applied by the RF system 920 to the RF coil 928, or a separate local coil to perform the prescribed magnetic resonance pulse sequence. Responsive magnetic resonance signals detected by the RF coil 928, or a separate local coil, are received by the RF system 920. The responsive magnetic resonance signals may be amplified, demodulated, filtered, and digitized under direction of commands produced by the pulse sequence server 910. The RF system 920 includes an RF transmitter for producing a wide variety of RF pulses used in MRI pulse sequences. The RF transmitter is responsive to the prescribed scan and direction from the pulse sequence server 910 to produce RF pulses of the desired frequency, phase, and pulse amplitude waveform. The generated RF pulses may be applied to the whole-body RF coil 928 or to one or more local coils or coil arrays.

The RF system 920 also includes one or more RF receiver channels. An RF receiver channel includes an RF preamplifier that amplifies the magnetic resonance signal received by the coil 928 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = ;$$

and the phase of the received magnetic resonance signal may also be determined according to the following relationship:

$$φ = {tan}^{- 1} .$$

The pulse sequence server 910 may receive patient data from a physiological acquisition controller 930. By way of example, the physiological acquisition controller 930 may receive signals from a number of different sensors connected to the patient, including electrocardiograph (“ECG”) signals from electrodes, or respiratory signals from a respiratory bellows or other respiratory monitoring devices. These signals may be used by the pulse sequence server 910 to synchronize, or “gate,” the performance of the scan with the subject's heart beat or respiration.

The pulse sequence server 910 may also connect to a scan room interface circuit 932 that receives signals from various sensors associated with the condition of the patient and the magnet system. Through the scan room interface circuit 932, a patient positioning system 934 can receive commands to move the patient to desired positions during the scan.

The digitized magnetic resonance signal samples produced by the RF system 920 are received by the data acquisition server 912. The data acquisition server 912 operates in response to instructions downloaded from the operator workstation 902 to receive the real-time magnetic resonance data and provide buffer storage, so that data is not lost by data overrun. In some scans, the data acquisition server 912 passes the acquired magnetic resonance data to the data processor server 914. In scans that require information derived from acquired magnetic resonance data to control the further performance of the scan, the data acquisition server 912 may be programmed to produce such information and convey it to the pulse sequence server 910. For example, during pre-scans, magnetic resonance data may be acquired and used to calibrate the pulse sequence performed by the pulse sequence server 910. As another example, navigator signals may be acquired and used to adjust the operating parameters of the RF system 920 or the gradient system 918, or to control the view order in which k-space is sampled. In still another example, the data acquisition server 912 may also process magnetic resonance signals used to detect the arrival of a contrast agent in a magnetic resonance angiography (“MRA”) scan. For example, the data acquisition server 912 may acquire magnetic resonance data and processes it in real-time to produce information that is used to control the scan.

The data processing server 914 receives magnetic resonance data from the data acquisition server 912 and processes the magnetic resonance data in accordance with instructions provided by the operator workstation 902. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 914 are conveyed back to the operator workstation 902 for storage. Real-time images may be stored in a data base memory cache, from which they may be output to operator display 902 or a display 936. Batch mode images or selected real time images may be stored in a host database on disc storage 938. When such images have been reconstructed and transferred to storage, the data processing server 914 may notify the data store server 916 on the operator workstation 902. The operator workstation 902 may be used by an operator to archive the images, produce films, or send the images via a network to other facilities.

The MRI system 900 may also include one or more networked workstations 942. For example, a networked workstation 942 may include a display 944, one or more input devices 946 (e.g., a keyboard, a mouse), and a processor 948. The networked workstation 942 may be located within the same facility as the operator workstation 902, or in a different facility, such as a different healthcare institution or clinic.

The networked workstation 942 may gain remote access to the data processing server 914 or data store server 916 via the communication system 940. Accordingly, multiple networked workstations 942 may have access to the data processing server 914 and the data store server 916. In this manner, magnetic resonance data, reconstructed images, or other data may be exchanged between the data processing server 914 or the data store server 916 and the networked workstations 942, such that the data or images may be remotely processed by a networked workstation 942.

The present disclosure has described one or more preferred embodiments, and it should be appreciated that many equivalents, alternatives, variations, and modifications, aside from those expressly stated, are possible and within the scope of the invention.

1. A method for cerebrospinal fluid (CSF) flow imaging with a magnetic resonance imaging (MRI) system, the method comprising:
(a) acquiring magnetic resonance data from a subject with an MRI system, wherein the magnetic resonance data are acquired using a pulse sequence having velocity encoding gradients sufficient to encode slow flow of the CSF in the subject;
(b) reconstructing phase contrast images from the magnetic resonance data using a computer system;
(c) generating velocity images from the phase contrast images using the computer system, wherein the velocity images are indicative of the slow flow of the CSF in the subject;
(d) generating CSF flow data from the velocity images using the computer system, wherein the CSF flow data are indicative of at least one of flow velocity magnitude, flow velocity direction, or flow volume of the CSF in the subject; and
(e) outputting the CSF flow data using the computer system.

2. The method of claim 1, wherein the pulse sequence comprises a pulsed-gradient spin-echo pulse sequence with an echo-planar imaging readout.

3. The method of claim 1, wherein the CSF flow data includes a flow velocity direction map that indicates the flow velocity direction of the CSF in the subject.

4. The method of claim 3, wherein the flow velocity direction map is generated by generating a velocity vector field based on the velocity images and projecting the vector field onto a two-dimensional image plane.

5. The method of claim 1, wherein the CSF flow data includes a flow velocity magnitude map that indicates the flow velocity magnitude of the CSF in the subject.

6. The method of claim 5, wherein the flow velocity magnitude depicted in the flow velocity magnitude map is an absolute flow velocity magnitude.

7. The method of claim 1, further comprising removing background phase from the magnetic resonance data before reconstructing the phase contrast images from the magnetic resonance data.

8. The method of claim 7, wherein removing the background phase comprises independently removing spatially low-polynomial-order phase of each dynamic.

9. The method of claim 1, further comprising performing motion correction on the magnetic resonance data before reconstructing the phase contrast images from the magnetic resonance data.

10. The method of claim 1, wherein the pulse sequence is a multi-velocity encoding (VENC) pulse sequence and the magnetic resonance data are indicative of multiple different velocity encodings corresponding to multiple different flow velocities.

11. The method of claim 1, wherein the velocity encoding gradients encode slow flow of the CSF of 3 cm/s or less.

12. The method of claim 11, wherein the velocity encoding gradients encode slow flow of the CSF of 1 cm/s or less.

13. The method of claim 1, wherein the magnetic resonance data are acquired while the subject is performing a functional task and the CSF flow data are indicative of flow dynamics responsive to the subject performing the functional task.

14. The method of claim 1, wherein the magnetic resonance data are acquired while the subject is in a particular state of arousal and the CSF flow data are indicative of flow dynamics responsive to the state of arousal.

15. The method of claim 1, wherein the magnetic resonance data are acquired from a whole brain of the subject and the CSF flow data are indicative of slow CSF flow in a subarachnoid space.

16. The method of claim 1, further comprising retrospectively gating at least one of the phase contrast images or the velocity images based on the CSF flow data.

17. The method of claim 16, wherein retrospectively gating the at least one of the phase contrast images or the velocity images based on the CSF flow data comprises grouping the at least one of the phase contrast images or the velocity images into groups associated with a physiological status.

18. The method of claim 17, further comprising generating velocity maps for each group.

19. The method of claim 18, wherein the physiological status comprises at least one of a cardiac cycle or a respiratory cycle.

20. The method of claim 1, wherein the pulse sequence is a single-shot pulse sequence.

21. The method of claim 1, wherein the pulse sequence is a multi-shot pulse sequence.
